(19) United States
(12) Patent Application Publication
(10) Pub. No.: US 2014/0031636 A1
(43) Pub. Date: Jan. 30, 2014

(54) ELECTROCHEMICAL AND OPTICAL TEST SPECIMEN

(71) Applicants: Kun-Lieh WU, Miaoli County (TW); Chin-Chang Yang, Miaoli County (TW)

(72) Inventors: Kun-Lieh WU, Miaoli County (TW); Chin-Chang Yang, Miaoli County (TW)

(21) Appl. No.: 13/562,074

(22) Filed: Jul. 30, 2012

(51) Int. Cl. A61B 5/15; A61B 5/1455; A61B 5/1468

(52) U.S. Cl. 600/301

(57) ABSTRACT

An electrochemical and optical test specimen includes an electrochemical test portion and an optical test portion. The electrochemical test portion has an electrochemical blood sampling port. The optical test portion has an optical blood sampling port. The electrochemical test portion and optical test portion abut each other, and the electrochemical blood sampling port and optical blood sampling port are located on the same side. Thus one blood sampling operation at the electrochemical blood sampling port and optical blood sampling port can be finished simultaneously to perform electrochemical test and optical test at the same time. Therefore the advantages of the electrochemical and optical tests can be integrated to achieve higher accuracy and lower cost to meet test requirements.

### FIELD OF THE INVENTION

The present invention relates to a test specimen and particularly to a test specimen for testing iron deficiency anemia.

### BACKGROUND OF THE INVENTION

Conventional test specimens generally are divided into electrochemical test specimens and optical test specimens according to their test principles. They absorb test fluid such as blood, urine or the like, then are disposed into a dedicated instrument to test physiological data such as blood sugar, lipids, hemoglobin, cholesterol, blood platelet, uric acid or the like.

The optical test specimen can measure the hemoglobin in the blood to determine whether there is anemia. It adopts the principle of breaking the blood cells in the absorbed blood to detect the color of the hemoglobin. During test a light ray of a selected wavelength is projected via an instrument, and the OD value is measured. Through change of the OD value the concentration of the hemoglobin can be derived, then judge whether anemia happens. It's widely adopted in medical practice now and provides acceptable accuracy.

The electrochemical test specimen also can detect hemoglobin in the blood to determine whether there is iron deficiency anemia. It also adopts the principle of breaking the blood cells in the absorbed blood. Through the characteristics of containing iron in the hemoglobin, the iron can be oxidized with a reaction agent on the test specimen and reduced, then current alteration can be detected to derive the concentration of the hemoglobin to judge whether iron deficiency anemia takes place.

However, the aforesaid test methods are easily being interfered and result in inaccurate test outcomes. The main cause is that other substances in the blood could also be oxidized and reduced, such as Vitamin C, uric acid or the like. Before a patient's blood sample is taken, if the patient has consumed improper food, the test result could only serve for reference to merely make a preliminary understand of the iron content, and accurate confirmation of the iron deficiency anemia cannot be made. In other words, they can serve merely for preliminary test, and other test methods have to be taken to make accurate confirmation of the iron deficiency anemia.

Because the conventional methods cannot truly test the iron deficiency anemia, many hospitals nowadays have to rely on an expensive HPLC analyzer to make further test after the preliminary test. Hence the tests have to be made twice to confirm the iron deficiency anemia. It's costly and cannot fully meet use requirements.

### SUMMARY OF THE INVENTION

Therefore the primary object of the present invention is to provide a low cost test specimen that can test iron deficiency anemia to meet use requirements.

To achieve the foregoing object, the present invention provides an electrochemical and optical test specimen. It includes an electrochemical test portion and an optical test portion. The electrochemical test portion has an electrochemical blood sampling port. The optical test portion has an optical blood sampling port. The electrochemical test portion and optical test portion abut each other, and the electrochemical blood sampling port and optical blood sampling port are located on the same side.

With the electrochemical blood sampling port and optical blood sampling port located on the same side, one blood sampling operation at the electrochemical blood sampling port and optical blood sampling port can be finished simultaneously to perform electrochemical and optical tests at the same time. Therefore the advantages of the electrochemical and optical tests can be integrated to achieve higher accuracy and lower cost to better meet test requirements.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of a first embodiment of the invention.

FIG. 2 is an exploded view of the first embodiment of the invention.

FIG. 3 is a side view of the first embodiment of the invention.

FIG. 4 is a perspective view of a second embodiment of the invention.

FIG. 5 is an exploded view of the second embodiment of the invention.

FIG. 6 is a side view of the second embodiment of the invention.

### DETAILED DESCRIPTION OF THE PREFERRED EMBODIMENTS

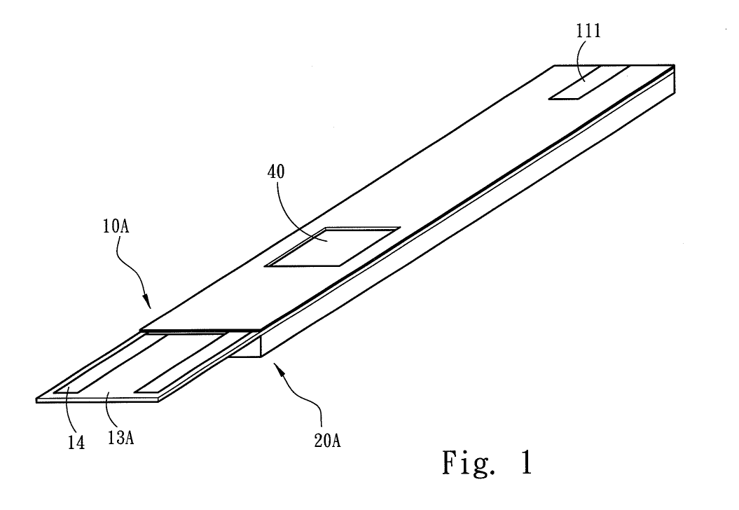

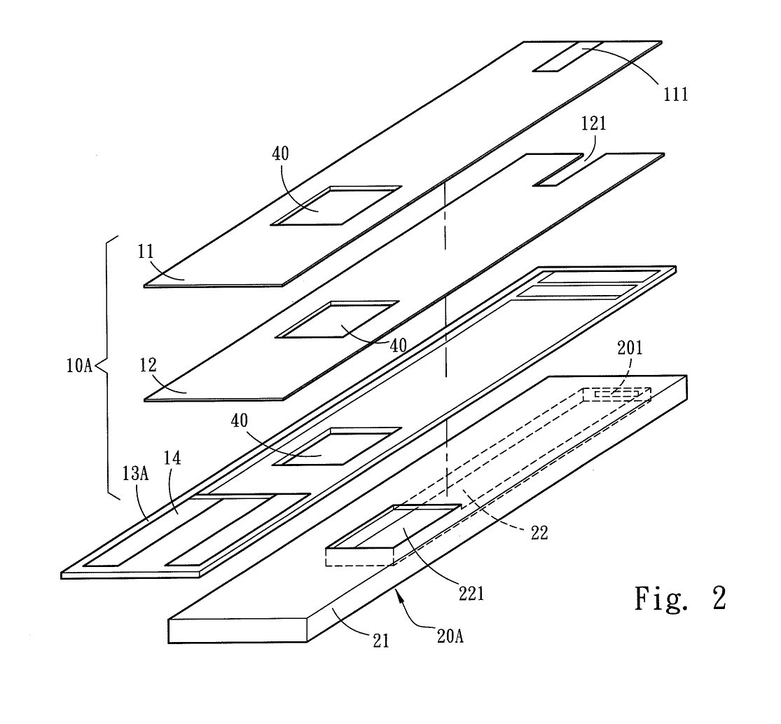

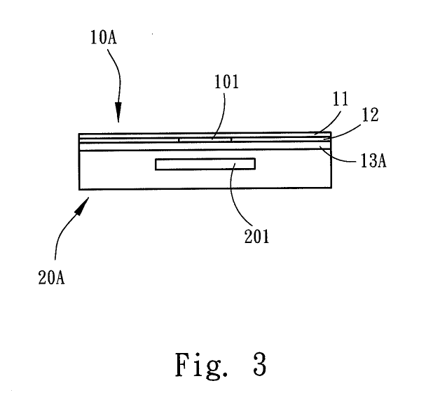

Refer to FIGS. 1, 2 and 3 for a first embodiment of the invention. The present invention aims to provide an electrochemical and optical test specimen. It includes an electrochemical test portion 10A and an optical test portion 20A. The electrochemical test portion 10A has an electrochemical blood sampling port 101. The optical test portion 20A has an optical blood sampling port 201. The electrochemical test portion 10A and optical test portion 20A abut each other, and the electrochemical blood sampling port 101 and optical blood sampling port 201 are located on the same side. The electrochemical test portion 10A and optical test portion 20A are stacked over each other in an abutting manner.

The electrochemical test portion 10A further includes an upper lid 11, a padded layer 12, a substrate 13A and a test circuit 14. The test circuit 14 is formed on the substrate 13A.

The upper lid 11 and substrate 13A are interposed by the padded layer 12 and bonded together. The padded layer 12 has a notch 121 to form the electrochemical blood sampling port 101. The upper lid 11 has a blood sampling viewing window 111.

The optical test portion 20A includes a reaction trough holder 21 which has a blood reaction notch 22 communicating with the optical blood sampling port 201. The blood reaction notch 22 has a transparent optical test window 221 remote from the optical blood sampling port 201. The substrate 13A, padded layer 12 and upper lid 11 have respectively a viewing window 40 corresponding to the optical test window 221.

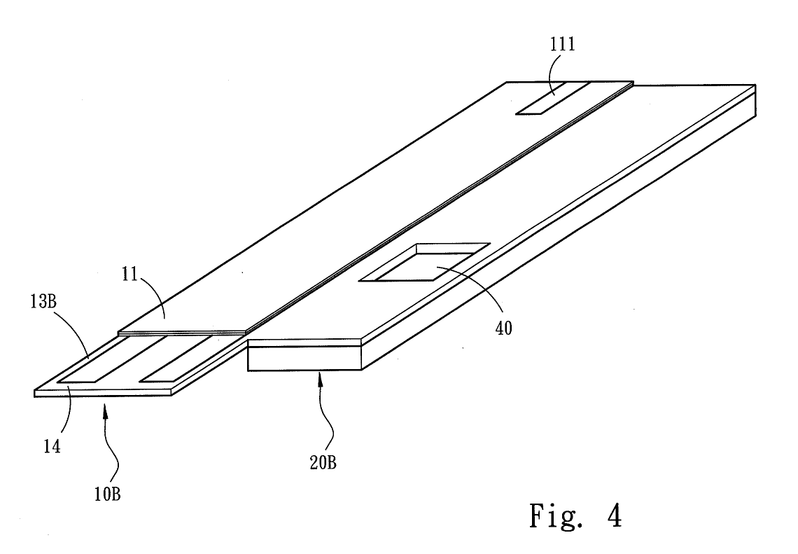

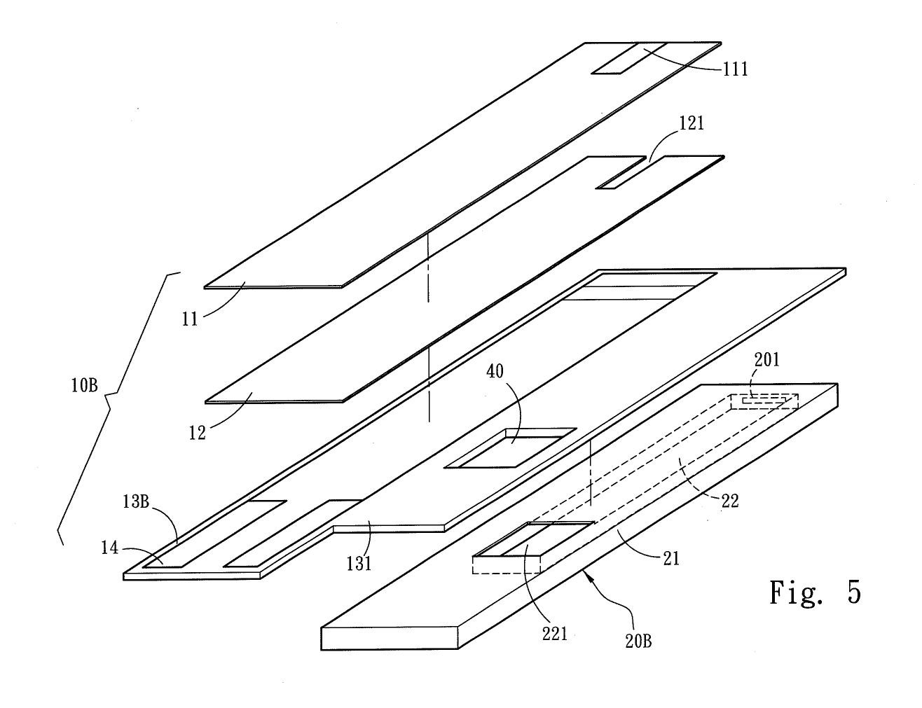

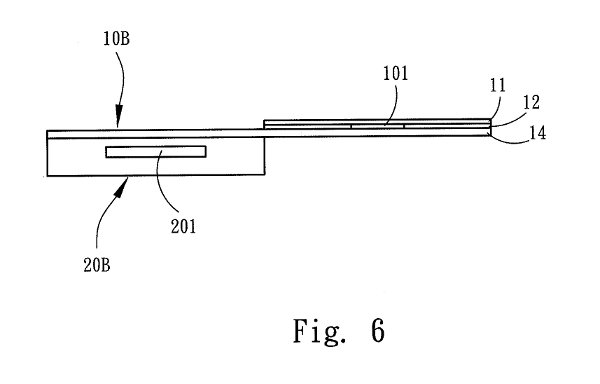

Please refer to FIGS. 4, 5 and 6 for a second embodiment of the invention. In this embodiment the electrochemical test portion 10B and optical test portion 20B are juxtaposed in an abutting manner. The electrochemical test portion 10B also has an upper lid 11, a padded layer 12, a substrate 13B and a test circuit 14. The test circuit 14 is formed on the substrate 13B. The upper lid 11 and substrate 13B are interposed by the padded layer 12 and bonded together. The padded layer 12 has a notch 121 to form the electrochemical blood sampling port 101. The upper lid 11 has a blood sampling viewing window 111.

The optical test portion 20B also has a reaction trough holder 21 which has a blood reaction notch 22 communicating with the optical blood sampling port 201. The blood reaction notch 22 has a transparent optical test window 221 remote from the optical blood sampling port 201. The substrate 13B is extended outwards to form a holding portion 131 which has a viewing window 40 corresponding to the optical test window 221. The holding portion 131 and reaction trough holder 21 are bonded together.

As previously discussed, by forming the electrochemical blood sampling port and optical blood sampling port on the same side, blood sampling can be finished in one blood sampling operation, thereby to perform electrochemical and optical tests at the same time, hence the advantages of the electrochemical and optical tests can be integrated.

Moreover, the invention can accurately test iron deficiency anemia. After the iron deficiency anemia has been tested via the electrochemical test, it then can be confirmed by the optical test. When both test results are positive, the iron deficiency anemia is confirmed. Hence there is no need to rely on the expensive HPLC analyzer to make further conformation. The cost is lower and accuracy is higher, and can fully meet test requirements.

What is claimed is:

1. An electrochemical and optical test specimen, comprising:
an electrochemical test portion including an electrochemical blood sampling port; and
an optical test portion which includes an optical blood sampling port located on the same side as the electrochemical blood sampling port and abuts the electrochemical test portion.

2. The electrochemical and optical test specimen of claim 1, wherein the electrochemical test portion and the optical test portion are stacked over each other in an abutting manner.

3. The electrochemical and optical test specimen of claim 2, wherein the electrochemical test portion includes an upper lid, a padded layer, a substrate and a test circuit; the test circuit being formed on the substrate, the upper lid and the substrate being interposed by the padded layer and bonded together, the padded layer including a notch to form the electrochemical blood sampling port.

4. The electrochemical and optical test specimen of claim 3, wherein the upper lid includes a blood sampling viewing window corresponding to the notch.

5. The electrochemical and optical test specimen of claim 3, wherein the optical test portion includes a reaction trough holder which has a blood reaction notch communicating with the optical blood sampling port, the blood reaction notch including a transparent optical test window remote from the optical blood sampling port; the substrate, the padded layer and the upper lid including respectively a viewing window corresponding to the optical test window.

6. The electrochemical and optical test specimen of claim 1, wherein the electrochemical test portion and the optical test portion are juxtaposed in an abutting manner.

7. The electrochemical and optical test specimen of claim 6, wherein the electrochemical test portion includes an upper lid, a padded layer, a substrate and a test circuit; the test circuit being formed on the substrate, the upper lid and the substrate being interposed by the padded layer and bonded together, the padded layer including a notch to form the electrochemical blood sampling port.

8. The electrochemical and optical test specimen of claim 7, wherein the upper lid includes a blood sampling viewing window corresponding to the notch.

9. The electrochemical and optical test specimen of claim 7, wherein the optical test portion includes a reaction trough holder which has a blood reaction notch communicating with the optical blood sampling port, the blood reaction notch including a transparent optical test window remote from the optical blood sampling port, the substrate being extended outwards to form a holding portion which includes a viewing window corresponding to the optical test window, the holding portion and the reaction trough holder being bonded together.
